(19) United States
(12) Patent Application Publication
(10) Pub. No.: US 2006/0140595 A1
(43) Pub. Date: Jun. 29, 2006

(54) Device for fragrance generation with a steam production

(75) Inventors: Jens Grabowski, Wolfisheim (FR); Dirk Sand, Chemnitz (DE)

Correspondence Address: DUANE MORRIS, LLP;IP DEPARTMENT, 30 SOUTH 17TH STREET, PHILADELPHIA, PA, 19103-4196, US

(73) Assignee: Hansgrohe AG, Schiltach (DE)

(21) Appl. No.: 10/538,256

(22) PCT Filed: Dec. 9, 2003
(86) PCT No.: PCT/EP03/13926

§ 371 Date: Jan. 10, 2006

(30) Foreign Application Priority Data
Dec. 12, 2002 (DE) 102 59 260.8

(51) Int. Cl. A61L 9/03

(52) U.S. Cl. 392/386

(57) ABSTRACT

A sample embodiment of the invention allows creating a device (11) for generating fragrances for distribution in steam rooms. Steam is conducted from a steam generator (38) to a vessel (13) by a steam supply line (15). A receptacle (26) for scent carriers (28), which may, for example, be similar to a sieve, is arranged within the vessel. The hot steam liberates fragrances, or scents, from the scent carriers. A steam exhaust line (16) for transporting steam saturated with fragrances, or scents, from the vessel (13), to the steam room, extends from the former, into the latter. The vessel (13) may be sealed by a lid (30). Emplacing that device (11) at a readily accessible location and moderate height within a steam room will provide for convenient operation thereof.

### APPLICATION ARE AND STATE OF ART

The invention relates to a device for generating fragrances or scenting steam generated by a steam generator. In particular, that device is suitable for use in a sauna, steam room, or steam bath/shower stall.

Admixing fragrances with steam in the case of steam rooms is known. Various techniques are available for that purpose. For example, allowing fragrances, such as oils, or similar, to vaporize on the hot steam exit nozzle is known. There are also systems that inject fragrances into the steam line to the steam exit nozzle using a metering pump. However, such metering pumps are elaborate and expensive.

### THE PROBLEM AND THE SOLUTION

The invention is based on the problem of creating a device of the aforementioned type for generating fragrances that will allow evading the disadvantages of the state of the art and, in particular, will allow generating fragrances in a relatively simple manner, while providing high degrees of protection against risks of sustaining burn injuries caused by hot steam.

That problem is solved by a device having those features stated under claim 1. Beneficial and preferred embodiments of the invention are covered by the other claims, and will be described in detail below. The wording of the claims is herewith made part of the content of this description by way of expressed reference thereto.

According to the invention, a device for generating fragrances or scenting steam has a source of steam. The source of steam itself may be configured in a known manner, for example, may be a steam generator. A main steam line may extend from the source of steam to a steam outlet that extends into a steam room. The device also has a vessel, within which, or on which, a receptacle for scent carriers is arranged. Another steam line extends from the source of steam, into the vessel, in particular, extends up to the receptacle. The steam will thus heat the scent carriers in order to instigate, or enhance, the evolution of fragrance or scent.

Scent carriers may be present in, for example, solid form. Leaves, needles, dried blossoms, or other parts of plants are available as carriers. Liquid scent carriers, such a ethereal oils, or similar, may also be employed. Preferred embodiments of the invention for use with various types of scent carriers will be taken up in greater detail below.

In the case of one embodiment of the invention, steam may be conducted from the steam supply line, directly to the scent carriers, causing them to be enveloped in steam, which will be beneficial in the case of scent carriers that have been designed for such treatment, or that require direct contact with steam. The receptacle may be configured to be similar to a sieve or mesh, and thus permeable to steam, for that purpose. A steam exhaust line may exit from, or from within, the vessel, and transport away steam that has been saturated with fragrance, or scent, and conduct it into the steam room.

In the case of another embodiment of the invention, it may be provided that steam from the steam supply line is conducted to the receptacle from without only, without its coming into contact with the scent carriers, where the receptacle is configured to be permeable to steam. Fragrance, or scent, liberated from the scent carriers may then be either conducted directly into the steam room, or conducted therein via a roundabout path.

It will be beneficial if the steam supply line from the source of steam, to the vessel, is a bypass line. The majority of the steam may be conducted to the steam outlet via the main steam line and, for example, enter a steam room. Namely, it has been found that if scent carriers are subjected to steam under favorable conditions, a relatively small quantity of steam will be sufficient to liberate fragrance, or scent, from the scent carriers, and thus to entrain them in the steam, or to saturate the steam with fragrance, or scent. Admixing fragrance, or scent, in a steam room may be avoided. A main steam line will also be kept as simple as possible.

The steam generated may be conducted into the vessel such that it is directly, and immediately, fed to the receptacle for scent carriers, or to the scent carriers themselves, where it may enter along a direction leading into the receptacle's interior, preferably from below and directed upward. The hot steam will then rise on its own in order to liberate scent from the scent carriers.

The receptacle may be configured to be similar to a sieve, or mesh, which will be particularly beneficial in the case of solid scent carriers, for example, the aforementioned parts of plants. If configured in that manner, the receptacle will be permeable to steam and steam may flow both into the receptacle and, after having been saturated with scent, out of the receptacle and the vessel. One option is a receptacle similar to a sieve having a mesh pitch of around several millimeters.

It will also be beneficial if saturation of the steam with scent may be halted or inhibited. It will be beneficial if the steam supply line entering the vessel, or receptacle, may be capped, or shut off, for that purpose. One means of accomplishing that in this particular case is a separate cap, in particular, a cap for the steam supply line entering the vessel. Compared to capping the steam exhaust line, capping the steam supply line has the advantage that high pressures that might cause damage or personal injuries cannot arise. It will also be beneficial if such a cap may be operated from outside the vessel. The cap might, for example, be moved over an aperture on the steam supply line for that purpose, in which case, reaching into the vessel, or opening the vessel, will be unnecessary.

An insert having a baseplate may be provided within the vessel for that purpose. That baseplate may be situated slightly above the aperture on the steam supply line and have an aperture that is at least about as large as the aperture on the steam supply line. Rotating the insert will allow the aperture in the baseplate to cover the aperture on the steam supply line to varying degrees, which will allow closing off the steam supply line, where it will be beneficial if either the underside of the baseplate abuts against the upper edge of the aperture on the steam supply line, or the aperture thereon is both covered and essentially sealed when the assembly in the closed state. The insert should therefore be fastened directly to the vessel such that it will not be forced upward by steam pressure and uncover the aperture on the steam supply line.

A device for detenting a cap on the aperture on the steam supply line may also be provided. That detenting device may allow closing off that aperture in certain, tactically perceptible, stages.

It will also be beneficial if, in addition to the aperture on the steam supply line entering the vessel, a steam exhaust line exiting the vessel is also provided. The aperture for the latter may be situated below the aperture for the steam supply line, which will provide that all steam will be exhausted from the vessel. Furthermore, in the event that the aperture for the steam exhaust line and the steam exhaust line face/run downward, they may be utilized as a drain for draining off small particles emanating from the scent carriers. Alternatively, a stream of steam passing through the scent carriers may be obtained, where it will be beneficial if it flows in the vertical or horizontal direction.

The vessel may beneficially be sealed. A lid that may, in particular, be emplaced on an aperture on the top of the vessel, may be provided for that purpose. Although the lid may be provided with a latch, it will be beneficial if it may be released, which will, beneficially, allow providing a safety valve, in the sense that the lid will be raised slightly whenever a certain overpressure within the interior of the vessel is exceeded. If that may occur for some very low overpressure, then high overpressures will never build up. The far end of the exiting steam exhaust line, i.e., that end thereof that is situated in the steam room, is preferably either configured without any facilities for capping it, or arranged such that it cannot be capped.

The lid may have a broad rim that overhangs the cross-section, or an outer edge, of the vessel. That rim may also have a section with an increased wall thickness, which will provide that the temperature of the rim will be reduced, since it will be heated less by the steam within the vessel, which contacts at least the midsection of the underside of the vessel's lid.

The lid may also be convexly domed. One option is a lid having a laterally broadened section and an upper section with an increased wall thickness. A neck, or waist, may be provided between the lid's laterally broadened section and its lower section. Among other things, that neck, or waist, may reduce heat conduction into the lid's broadened section somewhat in order that the broadened section will not be as hot as the remainder of the lid and may be utilized for lifting the lid off the vessel.

In the case of one prospective embodiment of the invention, the width of the broadened section may be such that, although the broadened section may still be inserted into the interior of the vessel when the lid is inverted, it may, beneficially, abut against the vessel's inner surface, forming a tight seal therewith, thereby also sealing the vessel. The benefit of such a sealing action in the vicinity of the broadened section is that hot steam will then be unable to flow into the lid's upper section, i.e., flow into that section thereof that is relatively close to its rim. Its rim will thus remain cooler, and the lid may thus be removed by grasping its rim with a bare hand, without risking sustaining burn injuries.

The lid may have a sort of recess, or cavity, on its underside. That recess, or cavity, may terminate close to the upper surface of the lid, and the lid may have a thinner wall in that vicinity as a result thereof. If the lid is inverted and set onto the vessel, i.e., set onto the vessel with its broadened section inserted into the latter, the recess will be situated within the vessel and be relatively close to the steam supply line, which will allow dribbling aromatic oil, or similar, into the recess while the lid is inverted. The former will then reside in the recess as though it were in a bowl, which will cause it to be heated from below by hot steam exiting the steam supply line, and cause the scent to flow upward, straight into a room, or steam room. Such a lid will thus also be suitable for use in vaporizing liquid scent carriers, or very fine-grained, solid, scent carriers.

In the case of another embodiment of the invention, the lid may have a relatively flat, essentially smooth, spherical, top, which will provide that it may be grasped exclusively by its outer edge, where temperatures will most likely be so low that risks of sustaining burn injuries will not occur.

It will also be feasible to arrange a protrusion extending upward, or diagonally outward, on an arbitrary type of lid. That protrusion may be used as a handgrip for removing, or otherwise manipulating, the lid.

A lid, or cap, beneficially consists of a plastic that should be able to withstand high temperatures, but not have too high a thermal conductivity, since otherwise the lid would be heated to overly high temperatures by the hot steam within the closed vessel.

If it may be removed from the vessel, the insert, or receptacle, may be readily cleaned in order to remove residual scent carriers, or residues left behind by scent carriers. The same statement applies to the lid. The vessel may also be provided with a mounting device, with which it may, beneficially, be fastened to a surface, for example, a horizontal shelf, which should be arranged at a moderate height, since that will allow relatively conveniently reaching the vessel in order to either fill it with scent carriers or shut off a steam supply line. In the case of the prospective embodiments described above, where scented oil is inserted into their inverted lid, it will be beneficial if scent generation occurs at a location that is not overly remote from a location of the head of a person present in the steam room.

These, and other, features of the invention will be evident from the claims, the description, and the figures, where the individual features involved may represent either themselves alone, or several such in the form of subcombinations thereof that have been implemented in an embodiment of the invention, or in other fields, and may represent beneficial embodiments that are themselves patentable, for which patent protection is herewith claimed.

### BRIEF DESCRIPTION OF THE FIGURES

A sample embodiment of the invention is depicted in the figures, and shall be described in greater detail below. The figures depict:

FIG. 1 a vertically sectioned view of a device according to the invention for generating steam that has a convexly domed lid emplaced thereon,

FIG. 2 another configuration of the device shown in FIG. 1 having an inverted lid serving as a receptacle bowl emplaced thereon,

FIG. 3 a horizontally sectioned view of the device shown in FIG. 1, and

FIG. 4 a schematized representation of a steam generator of the device shown in FIG. 1 and steam lines to a steam room.

### DETAILED DESCRIPTION OF THE SAMPLE

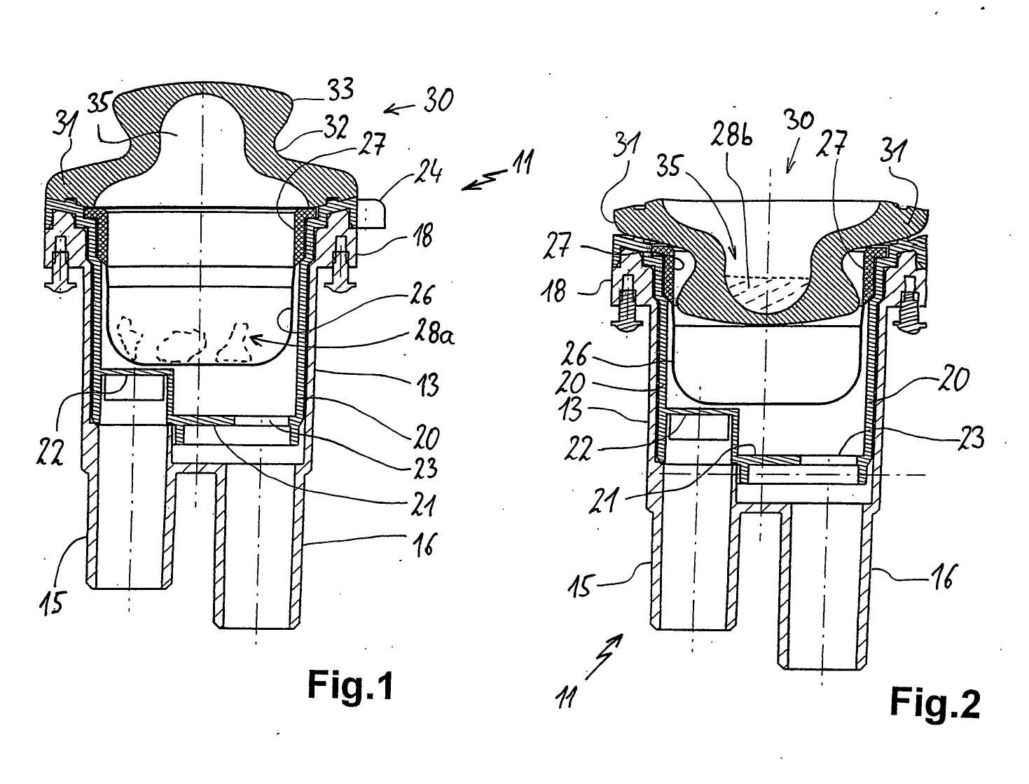

FIG. 1 depicts a device 11 according to the invention for generating fragrances, or for enriching steam for a steam room with scents, that essentially consists of a vessel 13 that may be fastened to a supporting surface 14, where it may be inserted into a mating aperture from above. The vessel 13 is roughly circular and cup-shaped. A flange 18 on the vessel overhangs a mating aperture in the supporting surface 14. The vessel 13 thus rests on the supporting surface 14 and may be bolted onto it from below, using the screws shown. The supporting surface 14 may, for example, be a pedestal, or a sort of sideboard, situated within a steam room, which has the major advantage that the device 11, or the vessel 13, will be readily reachable in order to, for example, place it in operation or refill it with scent carriers.

A steam supply line 15 enters the vessel 13 from below, and a steam exhaust line 16 exits it, where the steam exhaust line 16 commences at a location in the interior of the vessel below that where the steam supply line 15 terminates.

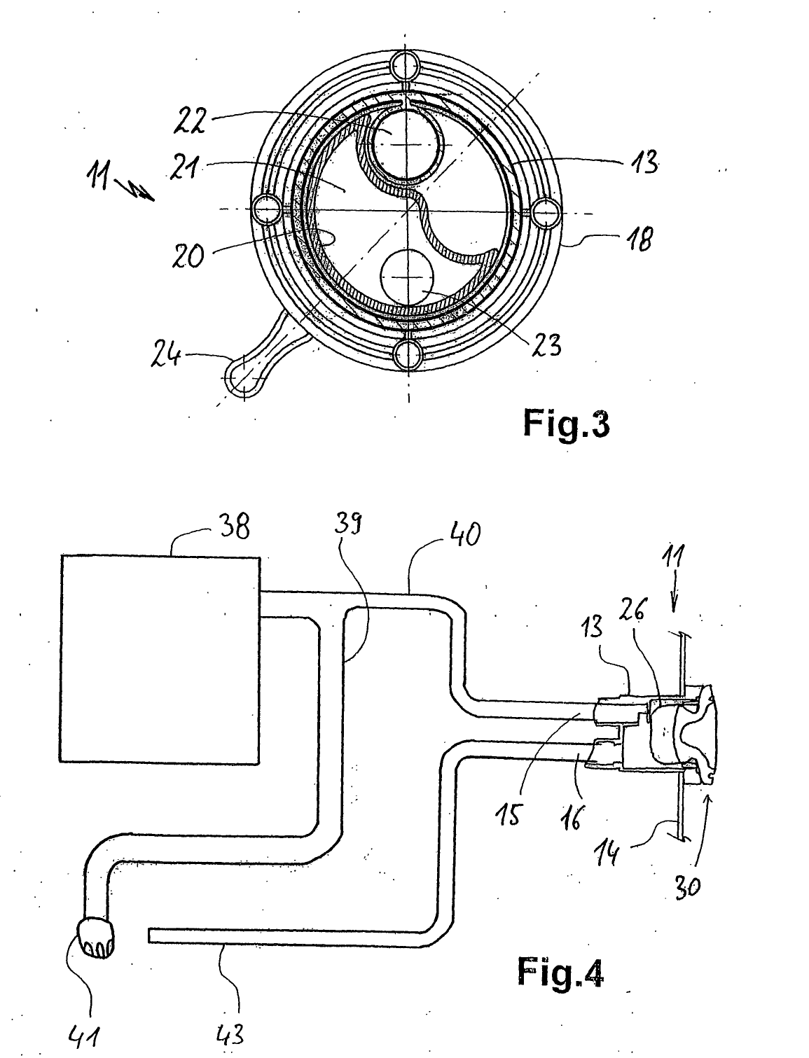

The vessel 13 contains an insert 20 that is also roughly circular and cup-shaped, where the insert 20 is essentially configured such that it conforms to the inner contours of the sidewall of the vessel 13. Its upper end has a multiply stepped rim that protrudes outward and has a lip extending downward. Its rim overlaps the flange 18 on the vessel. An adjustment prong 24 (cf. FIG. 3), with which the insert 20 may be rotated about a vertical axis while in the vessel 13, protrudes from the rim on the upper edge of the insert 20.

The insert 20 has a stepped base 21 (cf. FIGS. 1 and 2). The insert's base 21 is raised in the vicinity of the steam supply line 15, and has the aperture 22, through which steam from the steam supply line 15 enters the interior of the vessel 13. Another aperture 23 that leads to the steam exhaust line 16 is provided on the lower section of the insert's base 21. The extent to which the aperture 22 is circular, and thus essentially coincides with the steam supply line 15 when at a certain, single, position only, will be evident from FIG. 3. Rotating the insert will rotate the aperture 22 off the end of the steam supply line 15, thereby interrupting, or shutting off, the supply of steam to the vessel 13.

A carrier sieve 26 that is stepped in the vicinity of its upper edge 27 and accurately conforms to the contours of the inner wall of the insert 20 in that vicinity is also inserted into the vessel 13, or insert 20. The lower section of the carrier sieve 26 is configured in the form of a planar sieve, which is beneficial in the sense that certain solid scent carriers 28a, such as leaves or other parts of plants, may be held in the sieve 26, without falling through it. As may be seen, the lower section of the carrier sieve 26 extends nearly all the way down to the aperture 22 for the steam supply line 15, which will allow conducting steam straight into the carrier sieve 26, and to the scent carriers 28a situated therein, in which case, that section of the insert's base 21 where the aperture 22 is situated may also be shifted closer to the center of the carrier sieve 26, which will allow forcing more steam through all of the scent carriers situated therein.

A lid 30 rests atop the vessel 13. The lid 30 has a broad rim 31 that overhangs the internal cross-section of the vessel 13 and has a section having an increased wall thickness. Above its base, the lid 30 flares inward, forming a neck 32 that merges into a broadened section 33 serving as a sort of grip, where another, particularly large, increase in wall thickness is provided.

The inner wall of the lid is configured similarly to a bowl (cf. also FIG. 2). In particular, a recess 35 is situated within the broadened section 33 thereof. As may be seen from FIG. 2, that recess 35 extends fairly far into the interior of the vessel 13. For reasons that will be explained in greater detail below, the wall of the recess 35 is relatively thin at the lowest point thereof.

FIG. 4 depicts a schematized representation of a complete steam-generation facility. A main steam line 39 that terminates on a steam outlet 41 that, for example, terminates in the interior of a steam room, in particular, in the vicinity of its floor, exits a steam generator 38. Most of the steam is brought into the steam room by the main steam line 39.

A bypass steam line 40 that merges with the steam supply line 15 that terminates within the vessel 13 of the device 11 branches off from the main steam line 39. The steam exhaust line 16 exits from the vessel 13, and forms a fragrance-introduction line 43 that also terminates in the steam room. In the case of the example shown, it will also be feasible to have the fragrance-introduction line 43, which makes only a minor contribution to the formation of steam in the steam room, terminate higher up, which will facilitate fragrances reaching people who are sitting in the steam room.

As may be seen from FIG. 4, the bypass steam line 40 may be readily shut off, in particular, may be shut off using the insert's base 21 situated atop the steam supply line. Since steam is essentially capable of flowing out of the main steam line 39, no overpressure will build up in either the bypass steam line 40 or the steam supply line 15.

Remaining to be stated regarding the operation of the device 11 is that, in the case of a configuration like that shown in FIG. 1, solid scent carriers are placed in the carrier sieve 26. The lid 30 is then set in place and the insert 20 rotated to a position in which the inlet in the vessel 13 for admitting steam thereto via the steam supply line 15 is unobscured. Steam from the steam generator 38 will then flow through the bypass steam line 40 and into the vessel 13, where it comes into contact with the scent carriers 28a present in the carrier sieve 26. The hot and, under some circumstances, also moist, steam liberates the scents, or a fragrance, from the scent carriers, is exhausted through the steam exhaust line 16 and the fragrance-introduction line 43, and enters the steam room. If fragrance generation is to be discontinued, the only thing that needs to be done is rotating the insert 20 in the vessel 13 using the adjustment prong 24 thereon such that the steam supply line 15 will be blocked, in which case, namely, the aperture 22 therein will have been rotated away from that on the latter. That blocking of the steam supply line 15 may also be provided for use in cases where scent carriers are to be added or replaced.

The configuration shown in FIG. 2 is intended for use with scent carriers that have been configured for use at lower temperatures only or cannot be emplaced in the carrier sieve 26 because they are, for example, in liquid or powdered form. In such cases, the lid 30 is inverted and set onto the vessel 13, where the lid's broad rim 31 will invariably cover the interior of the vessel such that a sealing action will essentially occur. That sealing action will be aided by the lid's weight.

The broadened section 33 of the lid 30, i.e., that section thereof that has the recess 35 formed on its inner wall, will then protrude into the vessel 13. The bowl formed by the recess 35 may contain an aromatic oil 28b. Hot steam from the steam supply line 15 passes through the aperture 22 and enters the vessel 13 from below, and thus comes into contact with the underside of the lid 30. The steam heats the aromatic oil present in the recess 35 in a known manner. The fragrances and scents contained therein will thus be liberated and immediately thereafter enter the interior of the steam room. In this case, the wall of the lid 30 is thin in the vicinity of the bottom of the recess 35, which will allow attaining a good coupling of thermal energy into the aromatic oil 28b contained in the recess 35. The lid's wall thickness in that vicinity may be varied to suit the temperatures usually desired, or the steam temperatures or other circumstances or conditions involved, in order to affect the temperatures generated within the recess 35.

As shown in FIG. 2, the outer edge of the broadened section 33 on the lid 30 just clears the inner wall of the carrier sieve 26, a state of affairs that might be provided in the vicinity of the upper end of the latter in order to prevent hot steam from coming into contact with the rim 31 of the lid, which will essentially prevent the rim 31 of the lid from becoming too hot.

Slots, holes, or similar that may effect a particularly good thermal coupling of hot steam into the wall of the recess 35 may also be provided in the vicinity of the broadened section 33 of the lid 30. However, in the case of the configuration shown in FIG. 1, protrusions similar to cooling ribs provide for attaining particularly good heat transport from the lid 30 to the ambient.

Instead of such a lid 30 having a protruding, broadened section 33, a hemispherical lid whose shape roughly conforms to the lower section of the former type of lid 30 might be employed, in which case, the lid may be gripped by its rim only, which, as has been mentioned earlier, will usually not be as hot at the remainder of the lid. Protruding sections, similar to pins or handles, that would also allow safe handling of the lid, might also be provided.

In the event that powdered or liquid scent carriers 28a, 28b are to be placed in the vessel 13 itself and scent is to be transported out of the latter by the flow of steam, it will be feasible to, for example, remove the carrier sieve 26 and replace it with a differently configured receptacle, which may have an impermeable bowl. However, the receptacle should be heatable by the entering steam, and there should also be facilities for admixing scent emanating from scent carriers with the steam. In the case of, for example, a receptacle similar to the aforementioned carrier sieve 26, configuring an impermeable lower section would be sufficient for those purposes. An upper section, which might be configured such that it is permeable to steam or similar to a sieve, could allow steam to enter, entrain the liberated scent, and conduct it to the steam room via the steam exhaust line 16.

What is claimed is:

1. A device for generating fragrances comprising:
a steam generator,
a main steam line extending from the steam generator to a steam outlet, and
a vessel, within which a receptacle for scent carriers is arranged,
wherein a steam supply line from the steam generator is routed into the vessel and steam exiting therefrom heats the scent carriers for one of instigating and enhancing evolution of fragrance.

2. A device according to claim 1, wherein steam exiting the steam supply line is conducted directly to the scent carrier, where a steam exhaust line exits the vessel in order to conduct away steam that has been saturated with scent.

3. A device according to claim 2, wherein the receptacle comprises at least one of a sieve and a mesh that is permeable to steam.

4. A device according to claim 1, wherein steam from the steam supply line is conducted to the receptacle, without coming into contact with the scent carriers, and wherein the fragrance liberated from the scent carriers is conducted directly into the steam.

5. A device according to claim 1, wherein the steam supply line exiting the steam generator to the steam outlet comprises a bypass line, and wherein a greater part of the steam is conducted to the steam outlet by the main steam line.

6. A device according to claim 1, wherein the steam supply line is routed directly to the vessel and indirectly to the receptacle for scent carriers.

7. A device according to claim 1, having facilities for capping one of the steam supply line entering the vessel and the receptacle using a cap that is movable over an aperture on the steam supply line entering the vessel, from outside the vessel.

8. A device according to claim 1, further comprising an insert situated within the vessel that has a baseplate, wherein the baseplate overlaps the aperture on the steam supply line and has an aperture that can be brought into at least partial coincidence with the opening therein by rotating the baseplate.

9. A device according to claim 1, wherein a height of an aperture on the steam supply line entering the vessel exceeds that of an aperture on a steam exhaust line exiting the vessel.

10. A device according to claim 1, further comprising a lid for capping the vessel.

11. A device according to claim 10, wherein the lid has a broad rim that overhangs a cross-section of the vessel.

12. A device according to claim 10, wherein the lid is convexly domed and has a laterally broadened section and an increased wall thickness on its upper end.

13. A device according to claim 12, wherein the broadened section on the upper end of the lid is broad enough to extend into an interior of the vessel when the lid is inverted and placed atop the vessel.

14. A device according to claim 10 wherein the lid has a recess in its interior extending substantially up to the lid's upper surface.

15. A device according to claim 10 further comprising a prong that serves as a handgrip for use in removing the lid.

16. A device according to claim 1, wherein the receptacle is removable from the vessel.

17. A device according to claim 3, wherein the receptacle comprises a carrier sieve.

18. A device according to claim 4, wherein the receptacle is impervious to steam.

19. A device according to claim 6, wherein the steam supply line is directed toward an interior of the receptacle for scent carriers.

20. A device according to claim 8, further comprising a detenting device that can be preset by rotation through certain angles to adjust said aperture.

21. A device according to claim 10, wherein the lid is releasable at a certain overpressure.

22. A device according to claim 12, wherein a neck is formed between a laterally broadened section and a lower section of said lid.

23. A device according to claim 13, wherein the broadened section of the lid abuts against an inner surface of the vessel and forms a seal therewith.

24. A device according to claim 14, wherein the recess lies within the vessel when the lid is inverted and placed atop the vessel in order to form the receptacle.
